(19) United States
(12) Patent Application Publication
(10) Pub. No.: US 2020/0093814 A1
(43) Pub. Date: Mar. 26, 2020

(54) COMBINATION THERAPY WITH AMIDINE SUBSTITUTED BETA-LACTAM COMPOUNDS AND BETA-LACTAMASE INHIBITORS FOR INFECTIONS WITH ANTIBIOTIC RESISTANT BACTERIAL STRAINS

(71) Applicant: AiCuris GmbH & Co. KG, Wuppertal (DE)

(72) Inventors: Irith WIEGAND, Kaarst (DE); Cuong VUONG, Wuppertal (DE); Daniela M. RAUSCH, Wuppertal (DE)

(73) Assignee: AiCuris GmbH & Co. KG, Wuppertal (DE)

(21) Appl. No.: 16/471,677

(22) PCT Filed: Dec. 21, 2017
(86) PCT No.: PCT/EP2017/084064

§ 371 Date: Jun. 20, 2019

(30) Foreign Application Priority Data
Dec. 21, 2016 (EP) 16205800.2

(51) Int. Cl. A61K 31/454; A61K 31/427; A61K 45/06; A61K 31/424; A61K 31/431; A61K 31/43; A61P 31/04

(52) U.S. Cl. CPC A61K 31/454; A61K 31/427; A61K 45/06; A61P 31/04; A61K 31/431; A61K 31/43; A61K 31/424

(57) ABSTRACT

This invention relates to β-lactam compounds in combination with further drugs, e.g. β-lactamase inhibitors (BLIs), for use in the treatment and prophylaxis of infections caused by resistant bacteria.

### FIELD OF THE INVENTION

This invention relates to β-lactam compounds in combination with further drugs, e.g. β-lactamase inhibitors (BLIs), for use in the treatment and prophylaxis of infections caused by resistant bacteria.

### BACKGROUND

Public health experts and officials consider the emergence and spread of antibiotic resistant bacteria as one of the major public health problems of the 21st century. Although not a new phenomenon per se, the spread of antibiotic resistant bacteria has reached an unprecedented dimension. While the most resistant isolates continue to emerge in the hospital setting, physicians and epidemiologists are encountering increasing numbers of resistant bacteria in the community among people without previous healthcare contact.

The number of patients who are dying from untreatable nosocomial infections continues to grow. Therapeutic options are especially limited for infections due to multi-drug-resistant Gram-negative pathogens including Enterobacteriaceae and non-fermenters, a situation made worse by the fact that the pipelines of the pharmaceutical industry contain few compounds with promising resistance breaking profiles. There is a need to increase the number of effective antimicrobial drugs to defeat infections caused by bacteria that have become resistant to existing medicines [1].

Over the last few years, resistance rates for 3rd-generation cephalosporins steadily increased for invasive E. coli and K. pneumoniae isolates [2-4], primarily as a result of the rapid dissemination of plasmid mediated extended-spectrum β-lactamases (ESBLs) in hospitals which also hydrolyse 4- and 5-generation cephalosporins. ESBL are either variants of TEM- and SHV-type β-lactamases that were already prevalent among Enterobacteriaceae since the 1970s, or naturally endowed ESBL such as the CTX-M-enzymes. The latter have largely been replaced and outnumbered other types of ESBLs, but less frequent class A β-lactamases have also been described, including SFO, BES, BEL, TLA, GES, PER and VEB types [5]. Furthermore, cephalosporin resistance can be caused by overexpression of AmpC enzymes. AmpC β-lactamases are encoded in the genomes of various Enterobacteriaceae and non-fermenters such as Enterobacter species and P. aeruginosa and mutants with stably de-repressed AmpC production can be of clinical importance [6]. Additionally, ampC genes such as bla_{CMY} can also be found on plasmids.

Carbapenems are among the last options for treatment of severe infections caused by Gram-negative bacteria when cephalosporins are no longer reliable due to a high proportion of resistance; therefore, carbapenem resistance is a serious threat since it is leading to patient groups, for which few if any alternative treatment options are available [7].

In most European countries, the prevalence of resistance to carbapenems in Enterobacteriaceae is still generally low; however, the situation is different for some countries such as Greece, Italy, and Romania [4], where resistance in K. pneumoniae is increasing due to the spread of carbapenemases such as KPC- and also IMP-, VIM-β-lactamases, indicating their potential threat. Furthermore, outbreaks with carbapenemase-positive isolates (with KPC-, VIM-, IMP- or OXA-enzymes) of Enterobacteriaceae have been reported worldwide [9-11]. Infections caused by New Delhi metallo-(NDM) β-lactamase producers are relatively rare; however, these isolates are highly multi-resistant [12].

The co-production of β-lactamases from different groups and families in one single bacterial strain, can significantly broaden the spectrum of resistance to β-lactams. New β-lactams with resistance breaking properties are urgently needed to treat patients with infections caused by isolates producing multiple concomittant β-lactamases.

Aztreonam is the single FDA approved monobactam used worldwide and a second analogue is marketed exclusively in Japan (tigemonam). Reviews on aztreonam are available: W. C. Hellinger, N. S. Brewer; Carbapenems and Monobactams—Imipenem, Meropenem and Aztreonam; Mayo Clin. Proc. 1999, 74, 420-434. R. B. Sykes, D. P. Bonner; Discovery and Development of the monobactams; Rev. Infect. Dis. 1985, 7 (Suppl. 4), 579-593.

The attempt to enhance the cellular uptake of the β-lactams by using iron-siderophore uptake systems in microorganisms is one concept that has been explored in the monobactam field by Basilea (WO 2007065288), Naeja Pharmaceuticals (WO 2002022613) and Squibb & Sons (U.S. Pat. No. 5,290,929, EP 531976, EP 484881). The heteroaryl units mimicing siderophores can also be attached to the side-chain as hydrazides as demonstrated by Squibb & Sons (U.S. Pat. Nos. 5,318,963, 5,112,968).

Recently, Pfizer re-investigated monocarbams, mono-cyclic β-lactams that carry a sulfonylaminocarbonyl activating group at the Ni-position (WO 2010070523). Additionally, in WO 2008116813 Basilea has described combination therapy approaches using a combination of monobactams with carbapenems.

WO 2013 110643 describes β-lactam compounds of formula (I),

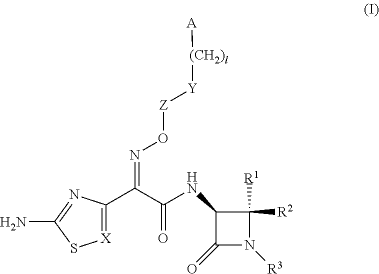

their preparation and use. In particular, WO 2013 110643 describes β-lactam compounds which are amidine substituted monobactam derivatives useful as antimicrobial agents and their preparation.

WO 2007 065288 describes a pharmaceutical composition comprising a combination of an antibiotically active compound of the formula (I) as depicted therein with β-lactamase inhibitors of one of the formulae (II) to (XIII), as depicted therein, for being active against Gram-negative bacteria, in particular such bacteria which have become resistant against antibiotics such as aztreonam, carumonam and tigemonam. Optionally, these compositions may comprise another β-lactamase inhibitor of one of the formulae (II) to (XIII) as depicted therein, particularly of formula (V) or formula (VI) as depicted therein.

In view of the increasing resistance development of pathogenic bacteria against known antibacterial agents, including multiple resistances, there is an urgent need to find new treatment options, in particular with useful combinations of drugs that upon coadministration as single drug products or as fixed combinations result in improved treatment or prophylactic effects.

### DISCLOSURE OF THE INVENTION

The present invention in one aspect relates to a compound of formula (I)

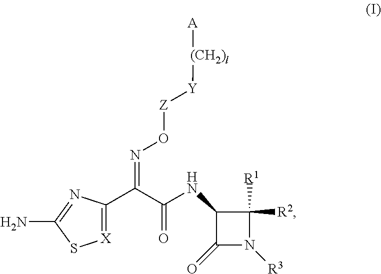

characterized in that
R1 and R2 represent methyl,
R3 represents —O—(SO₂)OH,
X represents CH,
Z represents a two carbon alkyl-chain, substituted with a carboxy substituent,
Y represents O,
A represents phenyl substituted with a substituent of the following formula

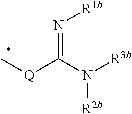

wherein
R1b and R2b represent hydrogen,
R3b represents aminoethyl, azetidine, pyrrolidine or piperidine,
Q represents a bond,
* is the linkage site to the residue represented by A, and
l represents 0,

and the salts thereof, the solvates thereof and the solvates of the salts thereof,

in combination with at least one β-lactamase inhibitor (BLI) selected from the group comprising clavulanic acid, tazobactam, sulbactam and other BLIs belonging, but not limited to, the groups of lactam inhibitors, diazabicyclooctane inhibitors, transition state analog inhibitors and/or metallo-β-lactamase inhibitors for use in the treatment or prophylaxis of a subject having an infection caused by Gram-negative bacteria that produce at least one class A and/or class D extended-spectrum β-lactamase (ESBL), selected from and not limited to the groups comprising TEM-ESBL, SHV-ESBL, OXA-ESBL, CTX-M-β-lactamases, SFO-, BES-, BEL-, TLA-, GES-, PER- and VEB-enzymes, and at least one additional β-lactamase selected from but not limited to the groups of class C AmpC β-lactamases and/or class A, class B, class C and/or class D carbapenemases, selected from and not limited to the group comprising IMP-, VIM-, SPM-, GIM-, SIM-, NDM-, OXA-, KPC-, GES-, SME-, IMI-, NMC-, FRI-, CcrA-, and PCD-β-lactamases.

In another aspect, the invention relates also to a compound as defined in the above aspect, which is selected from the group comprising compounds of formulae (I-a), (I-b), (I-c), (Id), (I-e), (I-f), and (I-g),

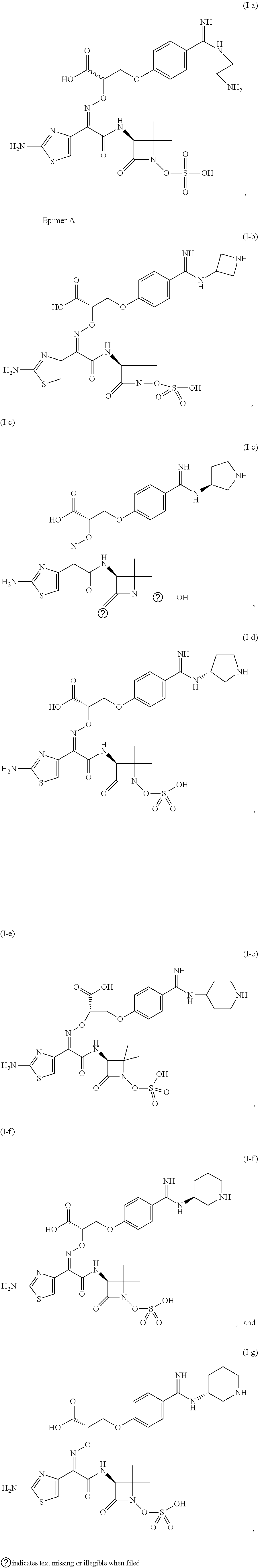

in combination with at least one β-lactamase inhibitor (BLI) selected from the group comprising clavulanic acid, tazobactam, sulbactam and other BLIs belonging to the groups of but not limited to lactam inhibitors, diazabicyclooctane inhibitors, transition state analog inhibitors and/or metallo-β-lactamase inhibitors for use in the treatment or prophylaxis of a subject having an infection caused by Gram-negative bacteria that produce at least one class A and/or class D extended-spectrum β-lactamase (ESBL) and at least one additional β-lactamase selected from the groups of but not limited to class C AmpC β-lactamases and/or class A, class B, class C and/or class D carbapenemases according to the preceding aspect, wherein the Gram-negative bacteria are selected from the genus of Enterobacteriaceae and non-fermenting Gram-negative bacteria.

In one aspect of the invention, the herein provided specific combinations of the general compound (I), as defined in the above aspect, and the specific compounds which are selected from the group comprising compounds of the formulae (I-a), (I-b), (I-c), (I-d), (Ie), (I-f), and (I-g) with only one specific BLI selected from the group comprising clavulanic acid, tazobactam, sulbactam and other BLIs belonging, but not limited to, the groups of lactam inhibitors, diazabicyclooctane inhibitors, transition state analog inhibitors and/or metallo-β-lactamase inhibitors are especially beneficial for its use in methods of prophylaxis and/or treatment against clinical strains that produce an ESBL and a second β-lactamase of a different class (i.e. so-called co-producers of more than one β-lactamase)-irrespective of the second β-lactamase type.

Specifically, with the context of the present invention, these said specific combinations with only one of the said BLIs is beneficial for use in the treatment or prophylaxis of a subject having an infection caused by Gram-negative bacteria that produce at least one class A and/or class D extended-spectrum β-lactamase (ESBL), selected from and not limited to the groups comprising TEM-ESBL, SHV-ESBL, OXA-ESBL, CTX-M-β-lactamases, SFO-, BES-, BEL-, TLA-, GES-, PER- and VEB-enzymes, and at least one additional β-lactamase selected from but not limited to the groups of class C AmpC β-lactamases and/or class A, class B, class C and/or class D carbapenemases, selected from and not limited to the group comprising IMP-, VIM-, SPM-, GIM-, SIM-, NDM-, OXA-, KPC-, GES-, SME-, IMI-, NMC-, FRI-, CcrA-, and PCD-β-lactamases.

For instance, and without being limited thereto, the specific combination of a compound (Ig) with e.g. clavulanic acid, or tazobactam or sulbactam alone shows improved in vitro and in vivo activity over known β-lactams in combinations with e.g. PIP, CAZ and AZT as BLI combination partners, which are yet on the market. According to the inventors' best knowledge, so far, there is no marketed monobactam combined with only one of the generic BLIs.

Hence, with the context of the present invention, especially the specific monobactam compound combinations with only one specific BLI selected from a group comprising clavulanic acid, tazobactam, sulbactam and other BLIs belonging, but not limited to, the groups of lactam inhibitors, diazabicyclooctane inhibitors, transition state analog inhibitors and/or metallo-β-lactamase inhibitors lead to a significantly improved activity against otherwise resistant co-producer strains.

With the above context, for instance, WO 2013 110643 describes the use of a compound (I-g) alone and in combination with clavulanic acid, and tazobactam and sulbactam in wild-type bacteria and bacteria producing only one β-lactamase (data are shown for E. coli TEM-3 producing one ESBL type β-lactamase in strain 18-21). But there is no data shown for highly resistant co-producer strains that produce more than one β-lactamase.

Hereto, the skilled artisan is well aware that activity against these highly resistant coproducers is not necessarily given and cannot be foreseen. This holds especially true since the state of the art literature best known to the instant inventors ever uses the combination of at least 2 or more active compounds to treat such co-producer strains that produce more than one β-lactamase.

For instance, WO 2008 116813 describes combination therapy approaches using a combination of monobactams with carbapenems (i.e. a combination of two β-lactams). U.S. Pat. No. 8,901,293 B2 or WO 2007 065288 describe only triple combinations of siderophore-monobactam, an AmpC inhibitor (so-called bridged monobactam) and a BLI, e.g. clavulanic acid, which demonstrate activity over referenced compounds therein (see Table 3, 4, 5 of WO 2007 065288).

Watkins et al. describe β-lactamase inhibitors with a therapeutic hope against the scourge of multidrug resistance (Front Microbiol. 2013; 4: 392). Therein, it is inter alia mentioned: “The report of a single isolate of K. pneumoniae producing a serine carbapenemase, a MBL, an ESBL and a plasmid-encoded AmpC carbapenemase underscores the challenge of using β-lactam antibiotics in the clinical setting (Pournaras et al., 2010). Treating this kind of pathogen with a β-lactam will likely require one with high stability to many common β-lactamases (e.g. aztreonam), together with two or more β-lactamase inhibitors that inhibit MBLs and serine β-lactamases. An example is the triple compound BAL30376”.

With the context of these specific aspects of the present invention, the inventors, however, surprisingly and unexpectedly inter alia found the compound (I-g) in combination with only one of the herein listed BLIs of the invention as already being active against highly resistant clinical co-producer strains that are simultaneously expressing more than one β-lactamase—irrespective of the BLI class itself.

Hence, by the specific combinations of the present invention no monobactam compound combinations with 2-fold, 3-fold or even 4-fold combinations of BLIs may be needed, even when a generic BLI is used as combination partner. According to the inventors' best knowledge this is against the general teachings and suggestions of the prior art.

In further aspects, the above described combinations may be used in combination with at least one other therapeutic agent as defined later in the treatment or prophylaxis of infections with antibiotic resistant bacteria.

By the term “combination” is meant either a fixed combination in one dosage unit form, or a kit or instructions for the combined administration where a compound of the present invention and a combination partner may be administered independently at the same time or separately within time intervals that especially allow that the combination partners show a cooperative, e.g., synergistic, effect, or any combination thereof.

The compounds of the present invention and/or the combination partner may, for example, be administered parenterally, pulmonarily, nasally, sublingually, lingually, buccally, rectally, dermally, transdermally, conjunctivally, otically or as an implant or stent.

Another possibility for the combined application of compounds of the present invention and combination partners is the co-drug approach where various effective drugs can be modified by attaching with other drugs of same or different categories directly or via a linkage [13].

The constituent drugs may be indicated for the same disease, but may exert different pharmacodynamic and/or pharmacokinteic therapeutic effects, e.g., an anti-infective therapeutic and/or prophylactic/preventive effect via disparate mechanisms of action. In the context of the present invention, an “anti-infective therapeutic and/or prophylactic/preventive effect” means that the compounds referred to herein have an effect on the vitality, reproducibility, infectivity, virulence, etc., or any other effect on a mechanism that induces, sustains, or worsens/deteriorates an infection with an infectious agent, e.g. any of the herein described bacteria.

In some embodiments, the therapeutic agent is an additional antibacterial agent.

Non-limiting examples of antibacterial agents for use in pharmaceutical combinations of the invention may be selected from but not limited to other clinically useful antibiotic agents such as penicillins, cephalosporins, penems, carbapenems, carbacephems (loracarbef and the like), oxacephems (moxalactam, latamoxef, flomoxef and the like), cephamycins (cefotetan and the like), monobactams, trinems (tritricyclic beta-lactams, tribactams and the like) aminoglycosides (tobramycin, gentamicin, amikacin, plazomicin and the like), bacteriocins (colicins, microcins and the like), quinolones (nalidixic acid and the like), fluoroquinolones (ciprofloxacin, moxifloxacin and the like), macrolides (erythromycin, roxithromycin, azithromycin and the like), ketolides (telithromycin and the like), tetracyclines (doxycycline, minocycline and the like), glycylcyclines (tigecycline and the like), oxazolidinones (linezolid, torezolid, radezolid and the like), lipopeptides (daptomycin and the like), polypeptides (actinomycin, bacitracin, and the like), polymyxins (colistin, polymyxin B and the like), rifamycins (rifampicin, rifabutin, rifapentine and the like), pleuromutilins (retapamulin, valnemulin, tiamulin, azamulin, lefamulin and the like), nitrofurans (nitrofurantion and the like), amphenicols (chloramphenicol, florphenicol and the like), nitroimidazoles (metronidazole and the like), glycopeptides (vancomycin and the like), lipoglycopeptides (oritavancin and the like), streptogramins (quinupristin, dalfopristin, pristinamycin derivatives and the like), ansamycins (streptovaricin derivatives and the like), lincosamides (lincomycin, clindamycin and the like), steroid antibacterials (fusidic acid and the like), folate pathway inhibitors (trimethoprim and the like), epoxyd antibacterials (fosfomycin and the like), nitroquinolines (nitroxoline and the like), antibacterial sulfonamides and antibacterial sulfathalidines (including para-aminobenzoic acid, sulfamethoxazole, sulfadiazine, sulfisoxazole, sulfathalidine and the like), xibornol and the like, clofoctol and the like, methenamine and the like, or derivatives thereof.

Pencillins include, but are not limited to, amdinocillin (mecillinam), amoxicillin, ampicillin, amylpenicillin, apalcillin, aspoxicillin, azidocillin, azlocillin, bacampicillin, carbenicillin, carindacillin, clometocillin, cloxacillin, cyclacillin (ciclacillin), dicloxacillin, epicillin, fenbenicillin, floxacillin (flucloxacillin), hetacillin, lenampicillin, metampicillin, methicillin, mezlocillin, nafcillin, oxacillin, penamecillin, penethecillin, penicillin G (procaine pencillin), penicillin N, penicillin O, penicillin V (phenoxymethyl penicillin), phenethicillin, piperacillin, pivampicillin, propicillin, quinacillin, sulbenicillin, talampicillin, temocillin, ticarcillin, pivmecillinam, benzathine penicillin, benzyl penicillin, or a combination thereof.

Cephalosporins include but are not limited to cephaloridin, cephradine, cefoxitin, cephacetril, cefoperazone, cefinenoxime, cephaloglycin, cefonicid, cefodizime, cefpirome, cefpiramide, cefozopran, cefoselis, cefluprenam, cefpimizole, cefclidin, cefpodoxime axetil, cefteram pivoxil, cefcapene pivoxil, ceftobiprole, ceftaroline, cefquinome, ceftiofur, cefovecin, cefadroxil, cefalonium, cefepime, cefotaxime, ceftazidime, cefetamet pivoxil, cefditoren pivoxil, cephaloridine, ceftazidime, ceftriaxone, cefbuperazone, cephalothin, cephazolin, cephapirin, ceftezole, cefamandole, cefotiam, cefotiam hexetil, cefuroxime, ceftizoxime, cefinenoxime, cefuzonam, cefsulodin, cefinetazole, cefminox, cephalexin, cefradine, cefaclor, cefadroxil, cefalonium, cefprozil, cefuroxime axetil, cefixime, cefpodoxime proxetil, ceftibuten, cefdinir, ceftolozane, or a combination thereof.

Penems include, without limitation, faropenem and carbapenems include, without limitation meropenem, ertapenem, doripenem, biapenem, panipenem, ritipenem, tebipenem, tomopenem, sulopenem, razupenem, imipenem, ME1036, SM216601 or a combination thereof.

Monobactams include, without limitation, aztreonam, carumonam, tigemonam, BAL19764, BAL30072 or a combination thereof.

Trinems (tritricyclic beta-lactams, tribactams) include, without limitation, GV104326 and the like or a combination thereof.

In some embodiments, the therapeutic agent is an additional drug selected from the group of but not limited to antifungal agents, antiviral agents, antiparasitic agents, antimycotic agents, antimycobacterial agents, intestinal antiinfective agents, biologicals (monoclonal antibodies, vaccines and the like), bactericidal/permeability-increasing protein product (BPI), antivirulence drugs, efflux pump inhibitors, probiotics, lysins, antimicrobial peptides, anti-biofilm agents, anti-resistance nucleic acids, anti-bacterial nucleic acids, antibiotic-degrading enzymes, alphamers, medical devices, antimalaria agents, antiinflammatory agents, antiallergic agents, centrally and peripherally acting analgesic drugs, anaesthetic drugs, immunomodulators, immune suppressive agents, monoclonal antibodies, anti-neoplastic drugs, anti-cancer drugs, anti-emetics, antidepressants, antipsychotics, anxiolytics, anti-convulsives, HMG CoA reductase inhibitors and other anti-cholesterol agents, anti-hypertensives, insulins, oral anti-diabetics, proton pump inhibitors, oral or parenteral anti coagulants, diuretics, digoxins, broncho dialators, anti-arrythmics, vasopressors, steroids and derivatives and combinations thereof.

Compounds of the formula (I) and (I-a to I-g) may also be co-administered with but not limited to the groups of penicillins, cephalosporins, penems, carbapenems, carbacephems, oxacephems, cephamycins, monobactams, trinems, tritricyclic beta-lactams, tribactams, aminoglycosides, bacteriocins, quinolones, fluoroquinolones, macrolides, ketolide, tetracyclines, glycylcyclines, oxazolidinones, lipopeptides, polypeptides, rifamycins, pleuromutilins, nitrofurans, amphenicols, nitroimidazoles, glycopeptides, lipoglycopeptides, streptogramins, ansamycins, lincosamides, steroid antibacterials, folate pathway inhibitors, epoxyd antibacterials, nitroquinolines, antibacterial sulfonamides and antibacterial sulfathalidines, xibornol, clofoctol, methenamine and the like, and derivatives thereof.

Compounds of the formula (I) and (I-a to I-g) may also contain or be co-administered with but not limited to antifungal agents, antiviral agents, antiparasitic agents, antimycotic agents, antimycobacterial agents, intestinal antiinfective agents, biologicals (monoclonal antibodies, vaccines and the like), bactericidal/permeability-increasing protein product (BPI), antivirulence drugs, efflux pump inhibitors, probiotics, lysins, antimicrobial peptides, anti-biofilm agents, anti-resistance nucleic acids, anti-bacterial nucleic acids, antibiotic-degrading enzymes, alphamers, medical devices, antimalaria agents, antiinflammatory agents, antiallergic agents, centrally and peripherally acting analgesic drugs, anaesthetic drugs, immunomodulators, immune suppressive agents, monoclonal antibodies, anti-neoplastic drugs, anti-cancer drugs, anti-emetics, antidepressants, antipsychotics, anxiolytics, anti-convulsives, HMG CoA reductase inhibitors and other anti-cholesterol agents, anti-hypertensives, insulins, oral anti-diabetics, proton pump inhibitors, oral or parenteral anti-coagulants, diuretics, digoxins, broncho dialators, anti-arrythmics, vasopressors, steroids and derivatives and combinations thereof.

The compound of formula (I) and (I-a to I-g) with a suitable therapeutic combination can be used for treating patients with bacterial infections, preoperative patients, postoperative patients, patients in intensive care unit (ICU), patients with nosocomial infections and veterinary infections.

The compounds described herein may be administered simultaneously, separately and/or sequentially with or without any medical device. In embodiments of the invention, the compounds of formula (I) and (I-a to I-g) and the BLI's referred to throughout the description may be administered at a ratio of 100:1 to 1:100, for example 10:1 to 1:10, such as 5:1 to 1:5. In a preferred embodiment, the compound is the compound (I-g).

Compounds for use according to the invention are the compounds of formula (I) or (I-a to I-g) and the salts, solvates and solvates of the salts thereof.

The herein described compounds of formula (I) or (I-a to I-g) and the salts, solvates and solvates of the salts thereof are for use in the treatment or prophylaxis of infections with resistant bacteria. According to embodiments of the present invention, the herein described compounds are for use in combination with a BLI as described in the preceding paragraphs.

In further aspects of the invention, the compounds of formulae (I) and (I-a to I-g) are used in the treatment or prophylaxis of infections with bacteria that belong to the group of ESBL producing bacteria, such as ESBL positive Enterobacteriaceae that are selected from the group comprising Citrobacter spp., Enterobacter spp., Escherichia spp. (e.g. Escherichia coli), Klebsiella spp. (e.g. Klebsiella pneumoniae), Proteus spp. (e.g. Proteus vulgaris, Proteus mirabilis), Providencia spp., Salmonella spp., Serratia spp. (e.g. Serratia marcescens), Yersinia spp., etc., wherein the compounds of formula (I) and (I-a to I-g) are combined with but not limited to oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), tribactams (GV104326 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, RG6080/OP0595, WCK 4234, WCK 5153, ETX-2514, CB-618 and the like), transition state analog BLIs (boronates, phosphonates, e.g. vaborbactam, MG96077 and the like), and/or metallo-β-lactamase inhibitors (e.g. captopril and the like).

In aspects of the combined use of compounds of formulae (I) and (I-a to I-g) with at least one of any BLIs selected from but not limited to the above compounds in the treatment or prophylaxis of infections with bacteria that belong to the group of ESBL-positive bacteria, the use of any of the compounds of formulae I-a, I-c, I-e, and/or (I-g) is explicitly contemplated. In a preferred embodiment, the compound is the compound (I-g).

The above-mentioned ESBL may be selected from the group comprising CTX-M, TEM, SHV, and VEB families.

In further aspects of the invention, the compounds of formulae (I) and (I-a to I-g) are used in the treatment or prophylaxis of infections with bacteria that belong to the group of KPC producing bacteria, wherein the compounds of formula (I) and (I-a to I-g) are combined with but not limited to oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), tribactams (GV104326 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, RG6080/OP0595, WCK 4234, WCK 5153, CB-618 and the like), transition state analog BLIs (boronates, phosphonates, e.g. vaborbactam, MG96077 and the like), and/or metallo-β-lactamase inhibitors (e.g. captopril and the like).

In further aspects of the invention the combined use of compounds of formulae (I) and (Ia to I-g) with any one of BLIs selected from the above compounds in the treatment or prophylaxis of infections with bacteria that belong to the group of KPC producing bacteria, the use of any one of the compounds of formulae I-a, I-c, I-e, and/or (I-g) is explicitly contemplated. In a preferred embodiment, the compound is the compound (I-g).

In further aspects of the invention, the compounds of formulae (I) and (I-a to I-g) are used in the treatment or prophylaxis of infections with bacteria that belong to the group of MBL producing bacteria, wherein the compounds of formula (I) and (I-a to I-g) are combined with but not limited to oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), tribactams (GV104326 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, RG6080/OP0595, NPI-1465, WCK 4234, WCK 5153, CB-618 and the like), transition state analog BLIs (boronates, phosphonates, e.g. vaborbactam, MG96077 and the like), and/or metallo-β-lactamase inhibitors (e.g. captopril and the like).

In further aspects of the invention, the combined use of compounds of formulae (I) and (I-a to I-g) with any one of the at least one additional BLI selected from the above compounds in the treatment or prophylaxis of infections with bacteria that belong to the group of MBL producing bacteria, the use of any one of the compounds of formulae I-a, I-c, I-e, and/or (Ig) is explicitly contemplated. In a preferred embodiment, the compound is the compound (I-g).

In further aspects of the invention, the compounds of formulae (I) and (I-a to I-g) are used in the treatment or prophylaxis of infections with bacteria that belong to the group of bacteria producing OXA-β-lactamases, wherein the compounds of formula (I) and (I-a to Ig) are combined with but not limited to oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), tribactams (GV104326 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, RG6080/OP0595, WCK 4234, WCK 5153, CB-618 and the like), transition state analog BLIs (boronates, phosphonates, e.g. vaborbactam, MG96077 and the like), and/or metallo-β-lactamase inhibitors (e.g. captopril and the like).

In further aspects of the invention, the combined use of compounds of formulae (I) and (I-a to I-g) with any one of the at least one additional BLI selected from the above compounds in the treatment or prophylaxis of infections with bacteria that belong to the group of OXA β-lactamase producing bacteria, the use of any one of the compounds of formulae I-a, I-c, I-e, and/or (I-g) is explicitly contemplated. In a preferred embodiment, the compound is the compound (I-g).

In further aspects of the invention, the compounds of formulae (I) and (I-a to I-g) are used in the treatment or prophylaxis of infections with bacteria that belong to the group of AmpC producing bacteria, wherein the compounds of formula (I) and (I-a to I-g) are combined with but not limited to oxapenams (e.g. clavulanic acid and the like), penam sulfones (e.g. tazobactam, sulbactam, AAI-101 and the like), bridged monobactams (e.g. BAL29880, MK-8712 and the like), monobactams (e.g. aztreonam, carumonam, tigemonam, BAL30072 and the like), tribactams (GV104326 and the like), cephem sulfones (e.g 7-alkylidenecephalosporin sulfone and the like), carbapenems (e.g. imipenem, meropenem, ertapenem, doripenem and the like), penems (e.g. LK-157 and the like), diazabicyclooctane inhibitors (e.g. avibactam, relebactam, zidebactam, RG6080/OP0595, WCK 4234, WCK 5153, CB-618 and the like), transition state analog BLIs (boronates, phosphonates, e.g. vaborbactam, MG96077 and the like), and/or metallo-β-lactamase inhibitors (e.g. captopril and the like).

In further aspects of the invention, the combined use of compounds of formulae (I) and (I-a to I-g) with any one of the at least one additional BLI selected from the above compounds in the treatment or prophylaxis of infections with bacteria that belong to the group of AmpC producing bacteria, the use of any one of the compounds of formulae I-a, I-c, I-e, and/or (I-g) is explicitly contemplated. In a preferred embodiment, the compound is the compound (I-g).

It is possible for each of the above aspects relating to the combined treatment of ESBL-, KPC-, MBL-, AmpC-positive bacteria, and OXA-carbapenemase producing bacteria to use more than one BLI selected from the group specifically referred to above. Further, it is possible to also treat affected subjects with additional medicaments that are selected from the group of but not limited to antibacterial agents, antifungal agents, antiviral agents, antiparasitic agents, antimycotic agents, antimycobacterial agents, intestinal antiinfective agents, biologicals (monoclonal antibodies, vaccines and the like), bactericidal/permeability-increasing protein product (BPI), antivirulence drugs, efflux pump inhibitors, medical devices, antimalaria agents, anti-inflammatory agents, antiallergic agents, centrally and peripherally acting analgesic drugs, anaesthetic drugs, immunomodulators, immune suppressive agents, monoclonal antibodies, anti-neoplastic drugs, anti-cancer drugs, anti-emetics, antidepressants, antipsychotics, anxiolytics, anti-convulsives, HMG CoA reductase inhibitors and other anti-cholesterol agents, anti-hypertensives, insulins, oral anti-diabetics, proton pump inhibitors, oral or parenteral anti coagulants, diuretics, digoxins, broncho dialators, anti-arrythmics, vasopressors, steroids and derivatives and combinations thereof.

The compounds of formulae (I) and (I-a to I-g) for use according to the invention may, depending on their structure, exist in stereoisomeric forms (enantiomers, diastereomers). The invention therefore also encompasses the enantiomers or diastereomers and respective mixtures thereof. The stereoisomerically uniform constituents can be isolated in a known manner from such mixtures of enantiomers and/or diastereomers.

If the compounds of formulae (I) and (I-a to I-g) for use according to the invention occur in tautomeric forms, the present invention encompasses all tautomeric forms.

The present invention also includes isotopically-labeled compounds, which are identical to those of formulae (I) and (I-a to I-g), but for the fact that one or more atoms are replaced by any atom having an atomic mass or mass number different from the atomic mass or mass number predominantly found in nature. Examples of isotopes that can be incorporated into compounds of the invention include but are not limited to isotopes of hydrogen, carbon, nitrogen, oxygen, such as, ²H, ³H, ¹³C, ¹⁴C, ¹⁵N, ¹⁸O, ¹⁷O, respectively.

Salts preferred for the purposes of the present invention are physiologically acceptable salts of the compounds of formulae (I) and (I-a to I-g) for use according to the invention. Also encompassed, however, are salts which are themselves not suitable for pharmaceutical applications but can be for use for example for the isolation or purification of the compounds of formulae (I) and (I-a to I-g) for use according to the invention. In a preferred embodiment, the salt is a physiologically acceptable salt of compound (I-g).

Examples of pharmaceutically acceptable salts [14] of the compounds of formulae (I) and (I-a to I-g) include salts of inorganic bases like ammonium salts, alkali metal salts, in particular sodium or potassium salts, alkaline earth metal salts, in particular magnesium or calcium salts; salts of organic bases, in particular salts derived from cyclohexylamine, benzylamine, octylamine, ethanolamine, diethanolamine, diethylamine, triethylamine, ethylenediamine, procaine, morpholine, pyrroline, piperidine, N-ethylpiperidine, N-methylmorpholine, piperazine as the organic base; or salts with basic amino acids, in particular lysine, arginine, ornithine and histidine. Examples of pharmaceutically acceptable salts of the compounds of formulae (I) and (I-a to I-g) for use according to the invention also include salts of inorganic acids like hydrochlorides, hydrobromides, sulfates, phosphates or phosphonates; salts of organic acids, in particular acetates, formates, propionates, lactates, citrates, fumarates, maleates, benzoates, tartrates, malates, methanesulfonates, ethanesulfonates, toluenesulfonates or benzenesulfonates; or salts with acidic amino acids, in particular aspartate or glutamate.

Solvates of formulae (I) and (I-a to I-g) for use for the purposes of the invention refer to those forms of the compounds of formulae (I) and (I-a to I-g) for use according to the invention which in the solid state forms a complex by coordination with solvent molecules. Hydrates are a specific form of solvates in which the coordination takes place with water.

The compounds of formulae (I) and (I-a to I-g) that are for use according to the invention show a valuable range of pharmacological effects which could not have been predicted.

They are therefore suitable for use as medicaments for the treatment and/or prophylaxis of diseases in humans and animals.

The compounds of the present invention are distinguished in particular by an advantageous range of antibacterial effects.

The present invention therefore further relates to the use of the compounds according to formulae (I) and (I-a to I-g) for use according to the invention for the treatment and/or prophylaxis of diseases caused by bacteria, especially Gram-negative bacteria. In embodiments of the invention, it is possible to use the compounds according to formula (I) and/or (Ia to I-g) in combination therapies as defined above also in the treatment and/or prophylaxis of suspected or verified infections with Gram-negative and Gram-positive bacteria, or in suspected or verified infections with Gram-negative bacteria and co-infections with viruses, fungi and/or parasites.

The present invention further relates to the use of the compounds according to formula (I) and/or (I-a to I-g) for use according to the invention for the treatment and/or prophylaxis of diseases, especially of the diseases mentioned below.

The present invention further relates to the use of the compounds according to formula (I) and/or (I-a to I-g) for use according to the invention for the manufacture of a medicament for the treatment and/or prophylaxis of diseases, especially of bacterial infections and in particular the diseases mentioned below.

The present invention further relates to methods for the treatment and/or prophylaxis of diseases, especially of bacterial infections and in particular the diseases mentioned below, using a therapeutically effective amount of the herein described combinations of compounds of formula (I) and/or (I-a to I-g) and BLIs as defined in any of the preceding embodiments of the invention. Further, it is possible to use the herein disclosed combinations of active compounds for the preparation of medicaments for the treatment and/or prophylaxis of a subject having an infection caused by Gram-negative bacteria that produce at least one or more class A and/or class D extended-spectrum β-lactamase (ESBL) and at least one additional β-lactamase selected from the groups of class C AmpC β-lactamases and/or class A, class B, class C or class D carbapenemases.

The compounds for use in methods of treatment or prophylaxis according to the above embodiments of the invention exhibit an antibacterial spectrum against Gram-negative bacteria combined with low toxicity.

Compounds of this invention are particularly useful in human and veterinary medicine for the prophylaxis and treatment of local and systemic infections which are caused for example by the following pathogens or by mixtures of the following pathogens: Aerobic Gram-negative bacteria: Enterobacteriaceae, including but not limited to Escherichia spp. (E. coli), Citrobacter spp. (C. freundii, C. diversus), Klebsiella spp. (K. pneumoniae, K. oxytoca), Enterobacter spp. (E. cloacae, E. aerogenes), Morganella morganii, Hafnia alvei, Serratia spp. (S. marcescens), Proteus spp. (P. mirabilis, P. vulgaris, P. penneri), Providencia spp. (P. stuartii, P. rettgeri), Yersinia spp. (Y. enterocolitica, Y. pseudotuberculosis), Salmonella spp., Shigella spp. and also non-fermenters including but not limited to Pseudomonas spp. (P. aeruginosa), Burkholderia spp. (B. cepacia), Stenotrophomonas maltophilia, and Acinetobacter spp. (A. baumannii, Acinetobacternosocomialis, Acinetobacter pitii) as well as Bordetella spp. (B. bronchiseptica), Moraxella catarrhalis and Legionella pneumophila; furthermore, Aeromonas spp., Haemophilus spp. (H. influenzae), Neisseria spp. (N. gonorrhoeae, N. meningitidis) as well as Alcaligenes spp. (including A. xylosoxidans), Pasteurella spp. (P. multocida), Vibro spp. (V. cholerae), Campylobacter jejuni and Helicobacter pylori.

Moreover, the compounds of the invention exhibit an antibacterial activity against strictly anaerobic bacteria including but not limited to Bacteroides spp. (B. fragilis), Peptostreptococcus spp. (P. anaerobius), Prevotella spp., Brucella spp. (B. abortus), Porphyromonas spp., and Clostridium spp. (Clostridium perfringens).

The compounds of the invention also exhibit an antibacterial activity against Gram-positive bacteria including but not limited to Staphylococcus spp. (S. aureus), Enterococcus spp., and Streptococcus spp. (S. pneumoniae, S. pyogenes, S. agalactiae, Streptococcus group C and G).

The above listing of pathogens is merely exemplary and in no way to be regarded as limiting.

Examples of diseases which may be caused by the said pathogens and which may be prevented, improved or cured by the compounds according to the invention are, for example: Respiratory tract infections such as lower respiratory tract infections, lung infection in cystic fibrosis patients, acute exacerbation of chronic bronchitis, community acquired pneumonia (CAP), nosocomial pneumonia (including ventilator-associated pneumonia (VAP)), diseases of the upper airways, diffuse panbronchiolitis, tonsillitis, pharyngitis, acute sinusitis and otitis including mastoiditis; urinary tract and genital infections for example cystitis, uretritis, pyelonephritis, endometritis, prostatitis, salpingitis and epididymitis; ocular infections such as conjunctivitis, corneal ulcer, iridocyclitis and postoperative infection in radial keratotomy surgery patients; blood infections, for example septicaemia; infections of the skin and soft tissues, for example infective dermatitis, infected wounds, infected burns, phlegmon, folliculitis and impetigo; bone and joint infections such as osteomyelitis and septic arthritis; gastrointestinal infections, for example dysentery, enteritis, colitis, necrotising enterocolitis and anorectal infections; intraabdominal infections such as typhoid fever, infectious diarrhea, peritonitis with appendicitis, pelviperitonitis, and intra-abdominal abscesses; infections in the oral region for example infections after dental operations; other infections for example, meliodosis, infectious endocarditis, hepatic abscesses, cholecystitis, cholangitis, mastitis as well as meningitis and infections of the nervous systems.

In addition to humans, bacterial infections can also be treated in animals, such as primates, pigs, ruminants (cow, sheep, goat), horses, cats, dogs, poultry (such as hen, turkey, quail, pigeon, ornamental birds) as well as productive and ornamental fish, reptiles and amphibians.

The compounds for use according to the embodiments of the invention may act systemically and/or locally. They can for this purpose be administered in a suitable way, such as, for example, parenterally, pulmonarily, nasally, sublingually, lingually, buccally, rectally, dermally, transdermally, conjunctivally, otically or as an implant or stent.

For these administration routes the compounds for use according to the invention can be administered in suitable administration forms, which are suitably prepared for use.

Parenteral administration can take place with avoidance of an absorption step (e.g. intravenous, intraarterial, intracardiac, intraspinal or intralumbar) or with inclusion of an absorption step (e.g. intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates or sterile powders.

Suitable for the other administration routes are, for example, pharmaceutical forms for inhalation (inter alia powder inhalers, nebulizers), nasal drops, solutions, sprays; tablets, films/wafers or capsules, for lingual, sublingual or buccal administration, suppositories, preparations for ears or eyes, vaginal capsules, aqueous suspensions (lotions, shaking mixtures), lipophilic suspensions, ointments, creams, transdermal therapeutic systems (such as for example patches), milk, pastes, foams, dusting powders, implants or stents.

The compounds according to formula (I) and/or (I-a to I-g) for use according to the invention can be converted into the stated administration forms. This can take place in a manner known per se by mixing with pharmacological inert, non-toxic, pharmaceutically acceptable excipients. These excipients include inter alia bulking agents (for example cyclodextrins, sorbitols, trehalose, lactose, mannitol PVP, amino acids, etc.), solvents (e.g. liquid polyethylene glycols, polysorbates, propylene glycols, alcohol), emulsifiers and dispersants or wetting agents (for example sodium dodecyl sulfate, polyoxysorbitan oleate, poloxomers), carriers (e.g. albumin), and stabilizers (e.g. antioxidants such as, for example, ascorbic acid).

The present invention further relates to medicaments which comprise at least one compound according to formula (I) and/or (I-a to I-g) for use according to the invention, usually together with one or more inert, non-toxic, pharmaceutically acceptable excipients, as well as to their use for the aforementioned purposes.

The minimum amount of the compounds according to formula (I) and/or (I-a to I-g) and the BLIs or other active compounds for use according to the invention to be administered is a therapeutically effective amount. The term “therapeutically effective amount” means an amount of compound which prevents the onset of, alleviates the symptoms of, stops the progression of, and/or eliminates a bacterial infection in humans or animals.

Typically, an effective dosing schedule of the compounds according to formula (I) and/or (I-a to I-g) for use according to the invention for adults is about 50 mg to about 5000 mg of a compound of formula (I) in a single dose; in another embodiment, an effective single dose is about 100 mg to about 3000 mg. In another embodiment, an effective single dose is about 500 mg to about 2000 mg. Dosages are usually given 1 to 4 times per day. In one embodiment, the dosages are given 3 times per day. In some cases, it may be necessary to use dosages outside these limits.

According to embodiments of the invention, wherein the BLI is selected from the group consisting of clavulanic acid, tazobactam, and sulbactam, the dose of clavulanic acid is 0.1-0.6 g, wherein the dose of tazobactam is 0.1-10 g, and wherein the dose of sulbactam is 0.25 g to 4.0 g, or preferably, wherein the dose of clavulanic acid is 0.25-0.6 g, wherein the dose of tazobactam is 0.25-2.0 g, and wherein the dose of sulbactam is 0.25 to 2.0 g.

In embodiments of the invention, the compounds of formulae (I) and (I-a to I-g) and the BLI's referred to throughout the description may be administered at a ratio of 100:1 to 1:100, for example 10:1 to 1:10, such as 5:1 to 1:5. In a preferred embodiment, the compound is the compound (I-g).

It may nevertheless be necessary, where appropriate, to deviate from the stated amounts, in particular as a function of body weight, administration route, individual response to the active ingredient, type of preparation and time or interval over which administration takes place.

Thus, in some cases it may be sufficient to make dosage with less than the aforementioned minimum amount, whereas in other cases the upper limit mentioned must be exceeded. In the case of an administration of larger amounts, it may be advisable to distribute these in a plurality of single doses over the day.

The percentage data in the following tests and examples are, unless indicated otherwise, percentages by weight; parts are parts by weight. Solvent ratios, dilution ratios and concentration data of liquid/liquid solutions are based in each case on volume. The statement “w/v” means “weight/volume”. Thus, for example, “10% w/v” means: 100 ml of solution or suspension contain 10 g of substance.

The herein described compounds of formula (I) and/or (I-a-g) may be produced and analyzed according to methods disclosed in WO 2013110643, particularly as described in the sections on “General Synthetic Methods” (pp. 42-244) and “Pharmacological Methods” (pp. 245-259), the contents of which are hereby incorporated by reference.

### EXAMPLES

Pharmacological Methods

Static Time Kill Curve

A liquid overnight culture of respective test strain was diluted in cation adjusted Mueller Hinton II (MH) broth with or without addition of physiological saline and grown until log phase growth was confirmed. The log phase culture was then diluted to approximately 1×10⁵ cfu/mL and 50 mL aliquots were prepared and maintained at 37° C., with 160 rpm shaking. At the zero time point (t=0 h), active substances were added to the cultures. At time points 0 h (negative control only), 1 h, 2 h, 4 h, 6 h, 8 h, 10 h and 12 h after treatment, samples were removed, serially diluted and plated onto Mueller Hinton agar plates to determine CFU counts. The experiments were carried out three times.

Minimum Inhibitory Concentration (MIC) Determination

Compounds of this invention were tested for antimicrobial activity by determining minimum inhibitory concentrations (MICs, in μg/mL) using the broth microdilution method according to the guidelines of the Clinical Laboratories and Standards Institute (Methods for Dilution Antimicrobial Susceptibility Tests for Bacteria that Grow Aerobically”, Approved standard, 7th ed., Clinical and Laboratory Standards Institute (CLSI) Document M7-A8, Wayne, Pa., USA, 2009.). While the compounds of this invention were serially diluted as described above, a constant concentration of the β-lactamase inhibitor of 4 ag/mL was used. Bacterial strains that were used to evaluate the antimicrobial activity using the MIC determination included but were not limited to E. coli 17, E. coli 72, E. coli 121, K. pneumoniae 79, E. coli 10, E. coli J62-TEM-3 transconjugant, E. coli 128, K. pneumoniae 68, K. pneumoniae 73, E. coli ATCC 25922, K. pneumoniae 137, K. pneumoniae 147, E. cloacae 58, K. pneumoniae CL5761, K. pneumoniae ATCC BAA-1898, K. pneumoniae ATCC BAA-1905, K. pneumoniae NRZ-01991, K. pneumoniae NRZ-15601.

Murine Peritonitis Infection Model

Murine infection experiments were performed as reported elsewhere [15]. Briefly, female CD-1 mice were randomized one day before infection to each treatment group of 5-6 animals. Mice were infected with a bacterial inoculum in the range of ˜1×10⁵ cfu-˜5×10⁸ cfu per mouse. Strains that were used included but were not limited to K. pneumoniae CL5761 (KPC-3 and SHV-5 co-producer) and E. cloacae 10⁵ (SHV-12). Compound (I-g) alone or in combination with at least one BLI was administered via i.v., i.p., or s.c. route in the tail vein to each mouse at 0.5-24 h post infection. No drug substance for treatment was administered to the negative, untreated but infected controls. For combination treatments, doses are given as dose for each active component. Surviving animals were monitored according to the institutional approval protocol and 4 times daily after 24 hours post infection for 120 hours. After 120 hours, all surviving animals were euthanized and antibacterial efficacy of the test substances was assessed by analysing the survival rates with Graph Pad Prism after 120 hours.

Preventive (Prophylactic) Murine Peritonitis Model

Briefly, female CD-1 mice were pretreated (i.v., i.p., s.c.) with compounds of this invention alone or in combination with at least one BLI prior to infection with a bacterial inoculum (˜1×10⁵ cfu-˜5×10⁸ cfu per mouse depending on the virulence of the strain used). Strains that were used included but were not limited to K. pneumoniae 68 (SHV-5) and K. pneumoniae CL5761 (KPC-3 and SHV-5 co-producer). Mice were euthanized 1 to 24 hours post-infection. Total blood was collected by terminal cardiac puncture and organs were aseptically removed, homogenized, diluted and plated on agar to determine the colony count (cfu/mL) per organ. Prophylactic effect and burden of bacterial dissemination was analyzed by comparing cfu counts between the treatment groups and control groups pretreated with 0.9% NaCl.

### Example 1

Activity of compound of formulae (I-a, I-c, I-e, and I-g) alone and in combination with clavulanic acid, tazobactam or sulbactam and reference antibiotics alone and in combination with clavulanic acid, tazobactam or sulbactam against ESBL producers with β-lactamases belonging to different families (Table 1). Aztreonam was chosen as reference for marketed monobactam antibiotics, ceftazidime as reference for marketed cephalosporins, and piperacillin as reference for marketed penicillin antibiotics (the latter is also available in combination with BLI tazobactam).

TABLE 1
Effect in ESBL producers

| | strain | | | | | | | | |
|---|---|---|---|---|---|---|---|---|---|
| | | | | | E. coli | | | | |
| | | | K. | | J62-TEM-3 | | K. | K. | E. coli |
| | E. coli | E. coli | pneumoniae | E. coli | trans- | E. coli | pneumoniae | pneumoniae | DSM |
| | 72 | 121 | 79 | 10 | conjugant | 128 | 68 | 73 | 22315 |
| | β-lactamase | | | | | | | | |
| | CTX-M | CTX-M | CTX-M | TEM-20 | TEM-3 | SHV-2a | SHV-5 | SHV-5 | VEB-1 |
| compound +/− BLI at 4 μg/mL | MIC (μg/mL) | | | | | | | | |
| piperacillin | >256 | >256 | >256 | >256 | 256 | >256 | >256 | >256 | >256 |
| ceftazidime | 32 | 32 | 16 | 32 | 16 | 16 | 64 | 256 | 128 |
| aztreonam | 128 | 64 | 32 | 64 | 8 | 8 | 128 | >256 | 64 |
| compound (I-g) | 0.25 | 0.25 | 0.25 | 2 | 2 | 1 | 8 | 16 | 8 |
| compound (I-a) | 0.125 | 0.25 | 0.25 | 2 | 4 | 1 | 8 | 16 | |
| compound (I-c) | 0.25 | 0.25 | 0.5 | 2 | 4 | 1 | 32 | 32 | |
| compound (I-e) | 0.25 | 0.25 | 0.5 | 2 | 2 | 1 | 16 | 16 | |
| piperacillin + clavulanic acid | 1 | 1 | 1 | 2 | 2 | 2 | 2 | 8 | 4 |
| ceftazidime + clavulanic acid | 0.25 | 0.125 | 0.125 | 0.25 | 0.25 | 0.25 | 0.125 | 0.5 | 0.25 |
| aztreonam + clavulanic acid | 0.125 | 0.125 | 0.031 | 0.063 | 0.125 | 0.063 | 0.063 | 0.125 | 0.125 |
| compound (I-g) + clavulanic | 0.031 | 0.031 | 0.063 | 0.031 | 0.063 | 0.031 | 0.125 | 0.25 | 0.031 |
| acid | | | | | | | | | |
| compound (I-a) + clavulanic | 0.031 | 0.063 | 0.063 | 0.031 | 0.063 | 0.063 | 0.125 | 0.25 | |
| acid | | | | | | | | | |
| compound (I-c) + clavulanic | 0.031 | 0.031 | 0.125 | 0.031 | 0.063 | 0.063 | 0.125 | 0.25 | |
| acid | | | | | | | | | |
| compound (I-e) + clavulanic | 0.063 | 0.031 | 0.063 | 0.031 | 0.063 | 0.063 | 0.063 | 0.5 | |
| acid | | | | | | | | | |
| piperacillin + tazobactam | 2 | 2 | 2 | 8 | 4 | >256 | 256 | 64 | >256 |
| ceftazidime + tazobactam | 0.5 | 0.25 | 0.25 | 0.25 | 0.5 | 8 | 4 | 8 | 4 |
| aztreonam + tazobactam | 0.125 | 0.125 | 0.063 | 0.125 | 0.125 | 4 | 4 | 8 | 4 |
| compound (I-g) + tazobactam | ≤0.031 | ≤0.031 | 0.125 | 0.063 | 0.063 | 0.25 | 0.5 | 1 | 0.25 |
| compound (I-a) + tazobactam | 0.031 | 0.031 | 0.063 | 0.031 | 0.125 | 0.25 | 1 | 2 | |
| compound (I-c) + tazobactam | 0.125 | 0.031 | 0.125 | 0.063 | 0.063 | 0.25 | 1 | 2 | |
| compound (I-e) + tazobactam | 0.063 | 0.063 | 0.063 | 0.063 | 0.063 | 0.25 | 1 | | |
| piperacillin + sulbactam | 256 | 128 | 16 | 128 | 2 | >256 | 256 | 256 | >256 |
| ceftazidime + sulbactam | 16 | 4 | 0.5 | 2 | 0.25 | 16 | 8 | >64 | 64 |
| aztreonam + sulbactam | 16 | 8 | 0.5 | 0.5 | 0.125 | 8 | 8 | >64 | 32 |
| compound (I-g) + sulbactam | 0.125 | 0.063 | 0.125 | 0.125 | 0.063 | 1 | 2 | 4 | 4 |
| compound (I-a) + sulbactam | 0.063 | 0.063 | 0.125 | 0.125 | 0.063 | 1 | 2 | 2 | |
| compound (I-c) + sulbactam | 0.063 | 0.063 | 0.125 | 0.125 | 0.063 | 1 | 2 | 2 | |
| compound (I-e) + sulbactam | 0.125 | 0.063 | 0.063 | 0.125 | 0.063 | 0.5 | 1 | 2 | |

### Example 2

Analysis of compounds (I-a, I-c, I-e, and I-g) combined with clavulanic acid, tazobactam or sulbactam in strains that produce ESBL and a second β-lactamase of a different class.

For clinical strains that produce ESBL and a second β-lactamase of a different class it was surprisingly found that irrespective of the second BL type combinations of the compound of formulae (I-a, I-c, I-e, and I-g)+tazobactam or the compound of formulae (I-a, I-c, I-e, and I-g)+clavulanic acid or the compound of formulae (I-a, I-c, I-e, and I-g)+sulbactam show lower MICs compared to the reference antibiotics combined with the respective BLIs.

TABLE 2
Effect in ESBL + AmpC (class C) co-producers

| | strain | | | |
|---|---|---|---|---|
| | K. pneumoniae 137 | K. pneumoniae 147 | E. cloacae 58 | E. coli 17 |
| | β-lactamase | | | |
| | SHV- | SHV- | SHV-12, de- | |
| | ESBL, | 12, | repressed | CTX-M, |
| | CMY-2 | CMY-2 | AmpC | CMY-2 |
| compound +/− BLI at 4 μg/mL | MIC (μg/mL) | | | |
| piperacillin | >256 | >256 | >256 | >256 |
| ceftazidime | 256 | 256 | 128 | 128 |
| aztreonam | 256 | >256 | 64 | 128 |
| compound (I-g) | 8 | 16 | 4 | 0.5 |
| compound (I-a) | 16 | 32 | 4 | 0.5 |
| compound (I-c) | 16 | >32 | 8 | 0.5 |
| compound (I-e) | 16 | 16 | 8 | 1 |
| piperacillin + clavulanic acid | 64 | 64 | 256 | 32 |
| ceftazidime + clavulanic acid | >64 | 64 | 64 | 32 |
| aztreonam + clavulanic acid | 64 | 64 | 32 | 8 |
| compound (I-g) + clavulanic acid | 2 | 2 | 0.5 | 0.125 |
| compound (I-a) + clavulanic acid | 2 | 2 | 1 | 0.125 |
| compound (I-c) + clavulanic acid | 2 | 2 | 1 | 0.125 |
| compound (I-e) + clavulanic acid | 2 | 2 | 0.25 | 0.125 |
| piperacillin + tazobactam | 64 | 64 | 256 | 32 |
| ceftazidime + tazobactam | >32 | >32 | >32 | 8 |
| aztreonam + tazobactam | 32 | 32 | 32 | 8 |
| compound (I-g) + tazobactam | 2 | 1 | 0.5 | 0.125 |
| compound (I-a) + tazobactam | 1 | 4 | 0.5 | 0.25 |
| compound (I-c) + tazobactam | 2 | 1 | 0.5 | 0.25 |
| compound (I-e) + tazobactam | 2 | 1 | 0.25 | 0.125 |
| piperacillin + sulbactam | 64 | 64 | 256 | 128 |
| ceftazidime + sulbactam | 64 | 64 | >64 | 64 |
| aztreonam + sulbactam | 64 | >64 | 32 | 16 |
| compound (I-g) + sulbactam | 2 | 1 | 1 | 0.25 |
| compound (I-a) + sulbactam | 2 | 4 | 0.5 | 0.25 |
| compound (I-c) + sulbactam | 4 | 2 | 1 | 0.5 |
| compound (I-e) + sulbactam | 2 | 2 | 1 | 0.125 |

TABLE 3
Effect in ESBL + KPC (class A carbapenemase) co-producers

| | strain | | | |
|---|---|---|---|---|
| | K. pneumoniae | K. pneumoniae | K. pneumoniae | K. pneumoniae |
| | CL5761 | ATCC BAA-1898 | ATCC BAA-1905 | NRZ-01991 |
| | β-lactamase | | | |
| | KPC-3, | KPC-2, | KPC-2, | KPC-2, |
| | SHV-5 | SHV-12 | SHV-12 | SHV-12 |
| compound +/− BLI 4 μg/mL | MIC (μg/mL) | | | |
| piperacillin | >512 | >512 | >512 | >512 |
| ceftazidime | 256 | 256 | 256 | 512 |
| aztreonam | 1024 | >256 | 256 | >512 |
| compound (I-g) | 8 | 8 | 16 | 16 |
| compound (I-a) | 8 | 16 | 32 | |
| compound (I-c) | 8 | 16 | >32 | |
| compound (I-e) | 8 | 16 | 16 | |
| piperacillin + clavulanic acid | >512 | >512 | >512 | >512 |
| ceftazidime + clavulanic acid | 256 | 64 | 64 | 128 |
| aztreonam + clavulanic acid | >512 | >512 | >512 | >512 |
| compound (I-g) + clavulanic acid | 1 | 1 | 1 | 2 |
| compound (I-a) + clavulanic acid | 2 | | | |
| compound (I-c) + clavulanic acid | 2 | | | |
| compound (I-e) + clavulanic acid | 1 | 2 | 2 | nd |
| piperacillin + tazobactam | >512 | >512 | >512 | >512 |
| ceftazidime + tazobactam | 256 | 128 | 64 | 256 |
| aztreonam + tazobactam | >512 | >512 | >512 | >512 |
| compound (I-g) + tazobactam | 4 | 2 | 2 | 4 |
| compound (I-a) + tazobactam | 2 | | | |
| compound (I-c) + tazobactam | 8 | | | |
| compound (I-e) + tazobactam | 8 | | | |
| piperacillin + sulbactam | >256 | >256 | >256 | >256 |
| ceftazidime + sulbactam | >64 | >64 | >64 | >64 |
| aztreonam + sulbactam | >64 | >64 | >64 | >64 |
| compound (I-g) + sulbactam | 4 | 8 | 4 | 8 |
| compound (I-a) + sulbactam | 8 | | | |
| compound (I-c) + sulbactam | 4 | | | |
| compound (I-e) + sulbactam | 4 | | | |

TABLE 4
Effect in ESBL + OXA-48 (class D carbapenemase) co-producers

| | strain | |
|---|---|---|
| | K. pneumoniae | K. pneumoniae |
| | 16/10 | NRZ-15601 |
| | β-lactamase | |
| | OXA-48, | OXA-48, |
| | CTX-M-15 | SHV-12 |
| compound +/− BLI at 4 μg/mL | MIC (μg/mL) | |
| piperacillin | >256 | |
| ceftazidime | 128 | 64 |
| aztreonam | >256 | 256 |
| compound (I-g) | 1 | 1 |
| compound (I-a) | 1 | |
| compound (I-c) | 1 | |
| compound (I-e) | 2 | |
| piperacillin + clavulanic acid | >256 | |
| ceftazidime + clavulanic acid | 32 | |
| aztreonam + clavulanic acid | >64 | |
| compound (I-g) + clavulanic acid | 0.5 | 0.25 |
| compound (I-a) + clavulanic acid | 0.5 | |
| compound (I-c) + clavulanic acid | 0.5 | |
| compound (I-e) + clavulanic acid | 0.5 | |
| piperacillin + tazobactam | >256 | 512 |
| ceftazidime + tazobactam | >32 | |
| aztreonam + tazobactam | >32 | |
| compound (I-g) + tazobactam | 0.5 | 0.25 |
| compound (I-a) + tazobactam | 2 | |
| compound (I-c) + tazobactam | 0.5 | |
| compound (I-e) + tazobactam | 1 | |
| piperacillin + sulbactam | >256 | >256 |
| ceftazidime + sulbactam | >64 | >64 |
| aztreonam + sulbactam | >64 | >64 |
| compound (I-g) + sulbactam | 2 | 0.5 |
| compound (I-a) + sulbactam | 1 | |
| compound (I-c) + sulbactam | 2 | |
| compound (I-e) + sulbactam | 1 | |

TABLE 5
Effect in ESBL + NDM (class B carbapenemase) co-producers

| | strain | | |
|---|---|---|---|
| | | | K. pneumoniae |
| | E. coli 2/10 | K. pneumoniae 601 | NCTC 13440 |
| | β-lactamase | | |
| | NDM-1, | NDM-1, | VIM-1, |
| | CTX-M-15 | CTX-M-15 | SHV-12 |
| compound +/− BLI at 4 μg/mL | MIC (μg/mL) | | |
| piperacillin | >256 | >256 | >256 |
| ceftazidime | >256 | >256 | >256 |
| aztreonam | >256 | >256 | 128 |
| compound (I-g) | 4 | 4 | 8 |
| compound (I-a) | 4 | 4 | |
| compound (I-c) | 4 | 4 | |
| compound (I-e) | 4 | 4 | |
| piperacillin + clavulanic acid | >256 | >256 | |
| ceftazidime + clavulanic acid | >64 | >64 | >32 |
| aztreonam + clavulanic acid | 32 | 16 | 0.063 |
| compound (I-g) + clavulanic acid | 1 | 1 | 0.125 |
| compound (I-a) + clavulanic acid | 1 | 0.5 | |
| compound (I-c) + clavulanic acid | 1 | 1 | |
| compound (I-e) + clavulanic acid | 1 | 0.5 | |
| piperacillin + tazobactam | >256 | >256 | >256 |
| ceftazidime + tazobactam | >256 | >256 | >32 |
| aztreonam + tazobactam | >32 | >32 | 0.25 |
| compound (I-g) + tazobactam | 1 | 2 | 0.125 |
| compound (I-a) + tazobactam | 2 | 2 | |
| compound (I-c) + tazobactam | 2 | 2 | |
| compound (I-e) + tazobactam | 1 | 4 | |
| piperacillin + sulbactam | >256 | >256 | |
| ceftazidime + sulbactam | >64 | >64 | |
| aztreonam + sulbactam | >64 | >64 | |
| compound (I-g) + sulbactam | 4 | 4 | 0.25 |
| compound (I-a) + sulbactam | 4 | 2 | |
| compound (I-c) + sulbactam | 4 | 2 | |
| compound (I-e) + sulbactam | 4 | 4 | |

### Example 3

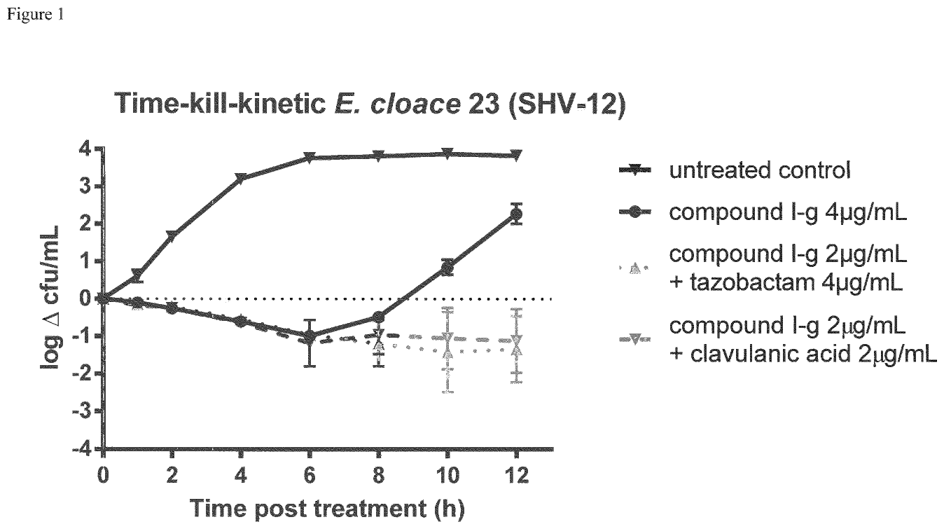

A time-kill kinetic with E. cloacae 23 (SHV-12) with compound I-g alone and in combination with tazobactam at 4 μg/mL or clavulanic acid at 2 μg/mL was made with the results shown in FIG. 1.

### Example 4

In vivo data (murine peritonitis infection model) is available for several strains of this collection (compound (I-g) alone and in combination with tazobactam or clavulanic acid).

i) Survival rates for animals treated with compound (I-g) alone and in combination with tazobactam or clavulanic acid, respectively, in a model of K. pneumoniae CL5761 (KPC-3 and SHV-5 co-producer) peritonitis infection.

Animals were infected via i.p. route with a bacterial inoculum in a range of 4.6-5.7×10⁷ cfu. Infected animals were treated with compound (I-g) at 3 mg/kg alone, compound (I-g) at 3 mg/kg in combination with tazobactam at 3 mg/kg, compound (I-g) at 3 mg/kg in combination with clavulanic acid at 1.5 mg/kg, or 0.9% sterile NaCl solution 0.5 hour, 1.0 hour and 2.0 hours post infection. Animals were monitored for survival up to 120 hours. In all experiments, 0.9% NaCl treated animals served as negative, untreated controls. Results are represented as Kaplan-Meier survival curves; differences in survival were calculated by the Log-rank test for negative, untreated control group vs. treated groups. At least three independent experiments were performed.

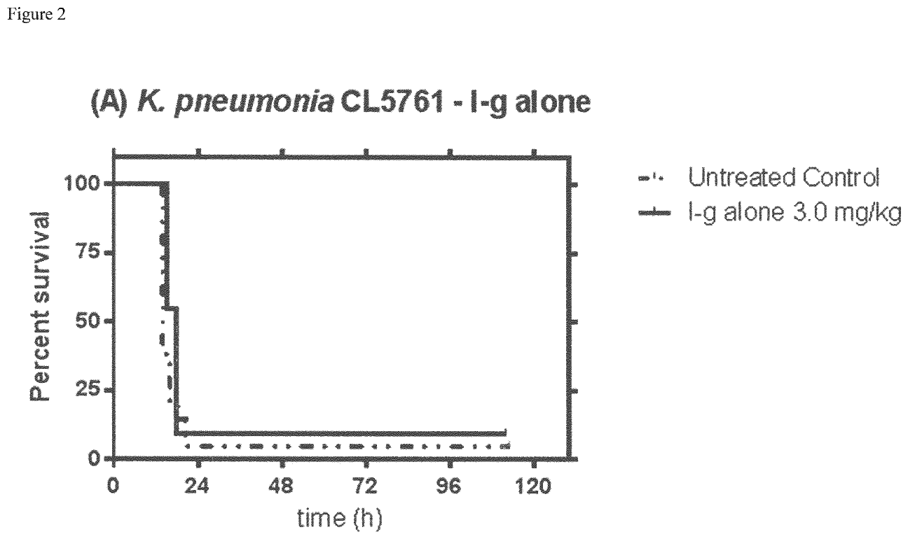

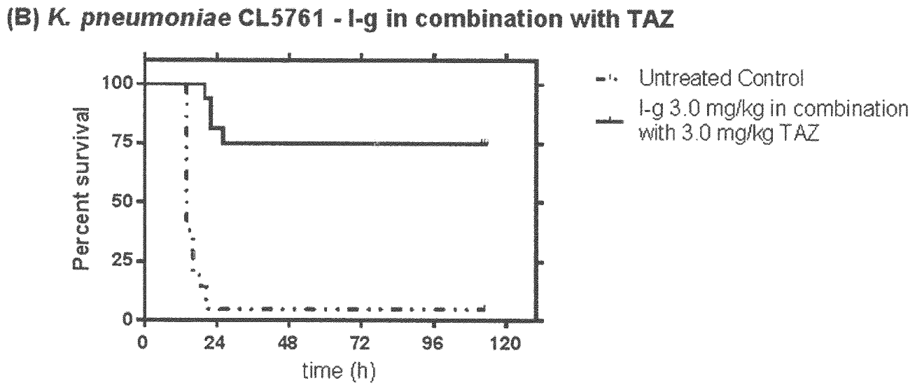

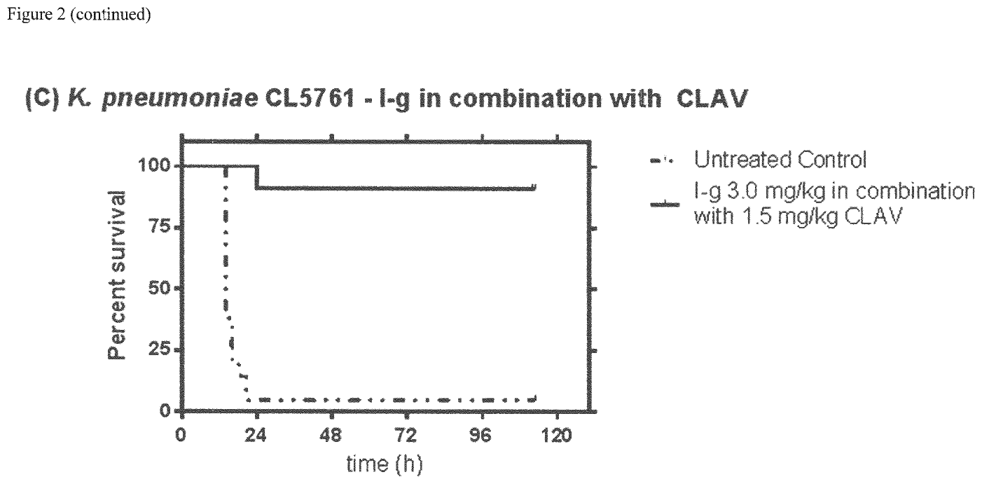

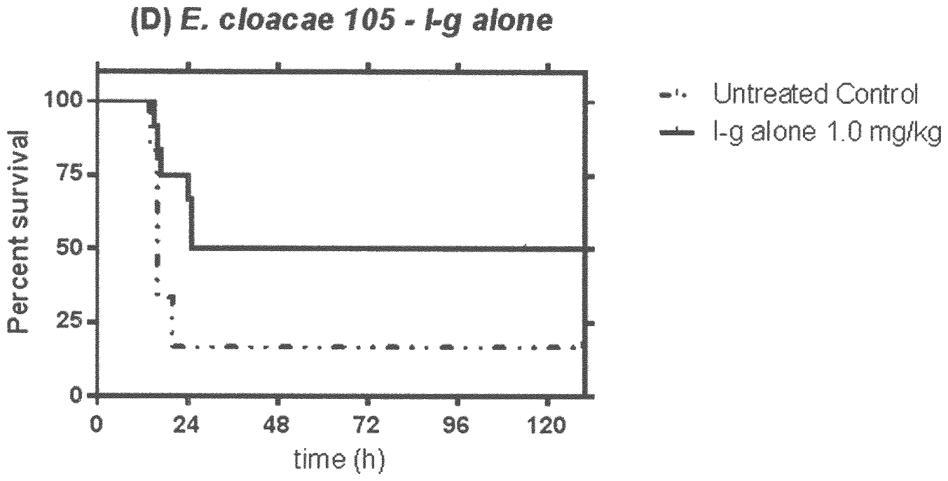

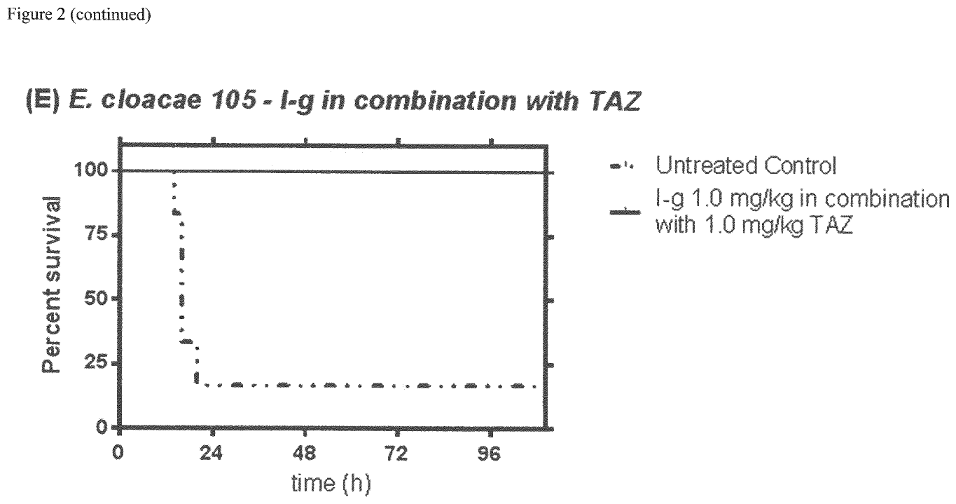

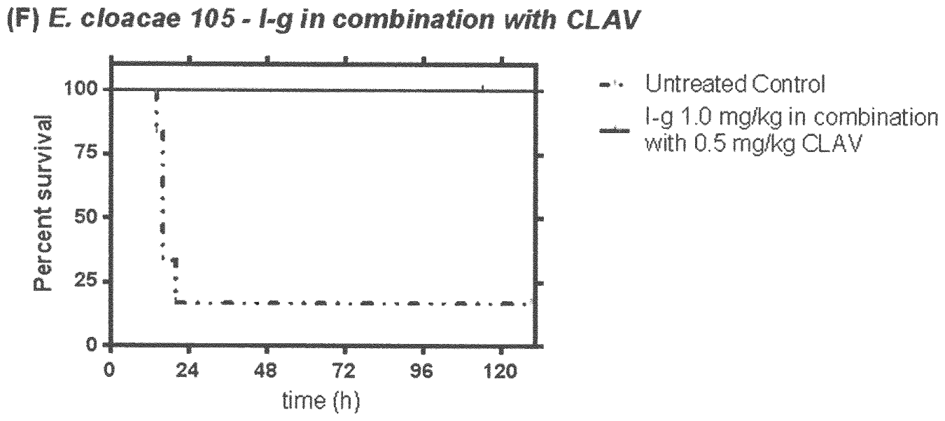

FIG. 2 shows the results: (A) 3.0 mg/kg compound (I-g) alone (ns−p=0.1686); (B) 3.0 mg/kg compound (I-g) in combination with 3.0 mg/kg tazobactam (p<0.0001); (C) 3.0 mg/kg compound (I-g) in combination with 1.5 mg/kg clavulanic acid (p<0.0001).

ii) Survival rates for animals treated with compound (I-g) alone and in combination with tazobactam or clavulanic acid, respectively, in a model of E. cloacae 105 (SHV-12) peritonitis infection.

Animals were infected via i.p. route with a bacterial inoculum in a range of 6.2-1.7×10⁷ cfu. Infected animals were treated with compound (I-g) at 1.0 mg/kg alone, compound (Ig) at 1.0 mg/kg in combination with tazobactam at 1.0 mg/kg, compound (I-g) at 1.0 mg/kg in combination with clavulanic acid at 0.5 mg/kg, or 0.9% sterile NaCl solution 0.5 hour, 1.0 hour and 2.0 hours post infection. Animals were monitored for survival up to 120 hours. In all experiments 0.9% NaCl treated animals served as negative, untreated controls. Results are represented as Kaplan-Meier survival curves; differences in survival were calculated by the Log-rank test for negative, untreated control group vs. treated groups. At least three independent experiments were performed. FIG. 2 D)-F) show the results. (D) 1.0 mg/kg compound (I-g) alone (ns−p=0.0576). (E) 1.0 mg/kg compound (I-g) in combination with 1.0 mg/kg tazobactam (p=0.0001). (F) 0.5 mg/kg compound (I-g) in combination with 0.5 mg/kg clavulanic acid (p=0.0001).

### Example 5

Comparison of K. pneumoniae 68 cfu/mL values in blood and organs at 120 and 180 minutes post-infection by pre-treatment groups.

Groups of female CD-1 mice (20 g) were pre-treated with a single dose (s.c.) of 90 mg/kg of compound (I-g)+45 mg/kg clavulanic acid or 90 mg/kg of compound (I-g)+90 mg/kg tazobactam 30 min prior to i.v. infection with approximately 2.3×10⁹ cfu of strain K. pneumoniae 68 (SHV-5). After 120 min or 180 min post-infection, mice were euthanized according to the Institutional Guidelines. Total blood was collected by terminal cardiac puncture and organs (kidney, liver, lungs) were removed aseptically, homogenized, diluted and plated by spiral dilution approach on agar to determine the number of bacterial colony-forming units (cfu) per ml.

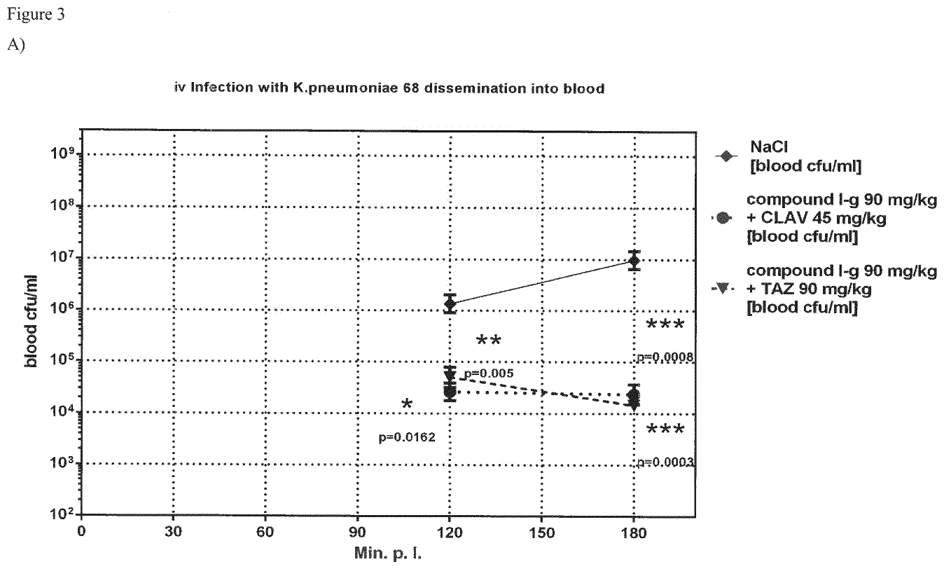

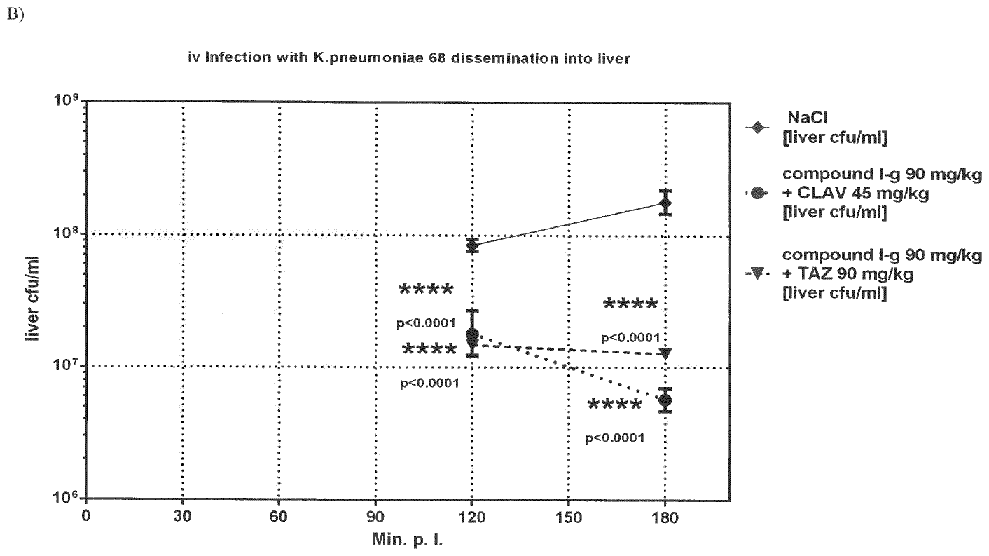

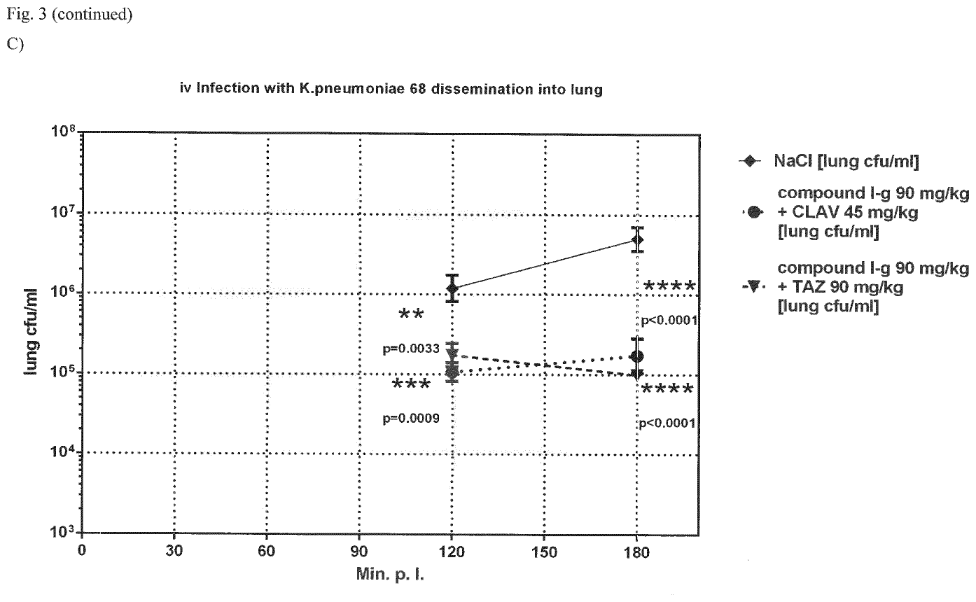

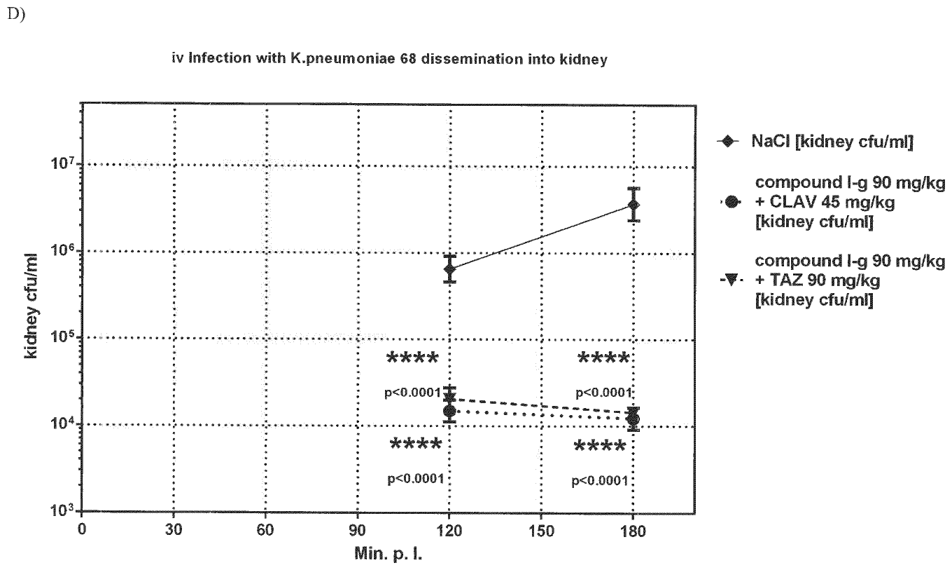

At 120 and 180 minutes p.i., geometric mean colony counts (cfu/mL) in all organs were statistically significantly lower in the compound (I-g) treated group either in combination with clavulanic acid or tazobactam, at both 120 and 180 minutes after infection, than in the control group which were pre-treated with NaCl. After 2 hours, the magnitude of reduction in the compound (I-g) group, either in combination with tazobactam or clavulanic acid, vs the NaCl group was generally approximately 10-fold, i.e., 1 log-point, which further increases to approximately 100-fold, i.e., 2 log-point after 3 hours post infection. FIG. 3A)-D) show the results; data points and lines represent the mean cfu/mL values with standard error for each treatment group

### REFERENCES

1. Jim O'Neill; The Review on antimicrobial resistance; Tackling drug-resistant infections globally, 2016
2. ECDC, Antimicrobial resistance surveillance in Europe 2012, in Annual Report of the European Antimicrobial Resistance Surveillance Network (EARS-Net). 2013, European Centre for Disease Prevention and Control: Stockholm.
3. ECDC, Antimicrobial resistance surveillance in Europe 2013, in Annual Report of the European Antimicrobial Resistance Surveillance Network (EARS-Net). 2014, European Centre for Disease Prevention and Control: Stockholm.
4. ECDC, Antimicrobial resistance surveillance in Europe 2014, in Annual Report of the European Antimicrobial Resistance Surveillance Network (EARS-Net). 2015, European Centre for Disease Prevention and Control: Stockholm.
5. Naas T., et al. Minor extended-spectrum β-lactamases. Clin Microbiol Infect. 2008. 14 (Suppl 1):p. 42-52
6. Jones, R. N., Important and emerging β-lactamase-mediated resistances in hospital-based pathogens: the Amp C enzymes. Diagn Microbiol Infect Dis, 1998. 31(3): p. 461-6.
7. Centers for Disease Control and Prevention, Antibiotic resistance threats in the United States, 2013. 2013, Centers for Disease Control and Prevention, US Department of Health and Human Services.
8. Espedido, B. A., et al., Whole genome sequence analysis of the first Australian OXA-48-producing outbreak-associated Klebsiella pneumoniae isolates: the resistome and in vivo evolution. PLoS One, 2013. 8(3): p. e59920.
9. Prevention, C.f.D.C.a., Vital signs: carbapenem-resistant Enterobacteriaceae. MMWR. Morbidity and mortality weekly report, 2013. 62(9): p. 165.
10. Glasner, C., et al., Carbapenemase-producing Enterobacteriaceae in Europe: a survey among national experts from 39 countries, February 2013. Euro Surveill, 2013. 18: p. 28.
11. Li, H., et al., Molecular characteristics of carbapenemase-producing Enterobacteriaceae in China from 2008 to 2011: predominance of KPC-2 enzyme. Diagn Microbiol Infect Dis, 2014. 78(1): p. 63-5.
12. Nordmann, P., et al., Does broad-spectrum beta-lactam resistance due to NDM-1 herald the end of the antibiotic era for treatment of infections caused by Gram-negative bacteria? J Antimicrob Chemother, 2011. 66(4): p. 689-92.
13. Das, N., et al., Codrug: An efficient approach for drug optimization. European Journal of Pharmaceutical Sciences, 2010. 41: p. 571-588.
14. Stahl, P H and Wermuth C G, Handbook of Pharmaceutical Salts. Willey-VCH 2012
15. N. Frimodl-Moller, J. D. Knudsen and F. Espersen: The mouse peritonitis/sepsis model. Chapter 14, p. 127-136. Handbook of animal models of infection. Academic press 1999.

### ABBREVIATION LIST

| Abbreviation | Explanation |
|---|---|
| AmpC | refers to a family of related |
| | enzymes, not to the same protein, produced in a variety of |
| | members of the family Enterobacteriaceae and non-fermenters |
| BEL | Belgium extended-spectrum β-lactamase |
| BES | Brazil extended spectrum |
| BLI | β-lactamase inhibitor |
| CcrA | carbapenem and cephamycin resistance |
| cfu | colony forming unit |
| CLAV | clavulanic acid |
| CLSI | Clinical and Laboratories Standard Institute |
| CMY | active on cephamycins |
| CTX-M | Active on cefotaxime, first isolated at Munich |
| ESBL | extended spectrum β-lactamase |
| FRI | French imipenemase |
| GES | Guiana extended spectrum β-lactamase |
| GIM | German imipenemase |
| h | hour |
| HMG CoA | Hydroxymethylglutaryl Coenzym A |
| IMI | imipenem-hydrolyzing β-lactamase |
| IMP | active on imipenem |
| i.p. | intraperitoneal |
| i.v. | intravenous |
| KPC | Klebsiella pneumoniae carbapenemase |
| MBL | metallo β-lactamase |
| MDR | multidrug resistant |
| MIC | minimal inhibitory concentration |
| min | minute |
| NMC | not metalloenzyme carbapenemase |
| NDM | New Delhi metallo β-lactamase |
| OAT | organic anion transporter |
| OXA | oxacillinase |
| PCD | Pseudomonas-derived cephalosporinase |
| PER | pseudomonas extended resistance |
| s.c. | subcutaneous |
| SHV | sulfhydryl variable |
| SIM | Seoul imipenemase |
| SME | Serratia marcescens enzyme |
| SPM | Sao Paulo metallo-β-lactamase |
| TAZ | tazobactam |
| TEM | lactamase named after the patient (Temoneira) providing the first sample |
| VEB | Vietnamese extended spectrum β-lactamase |
| TLA | lactamase named after the Tlahuicas Indians providing the first sample |
| VIM | Verona integron-encoded metallo β-lactamase |
| vs. | versus |

1. A composition comprising a compound of formula (I) 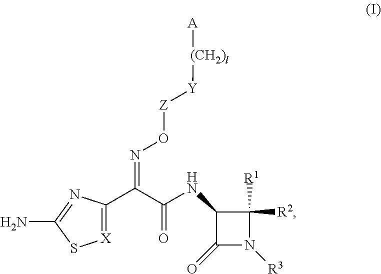
characterized in that
R1 and R2 represent methyl,
R3 represents —O—(SO₂)OH,
X represents CH,
Z represents a two carbon alkyl-chain, substituted with a carboxy substituent,
Y represents O,
A represents phenyl substituted with a substituent of the following formula 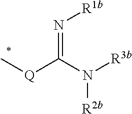
wherein
R1b and R2b represent hydrogen,
R3b represents aminoethyl, azetidine, pyrrolidine or piperidine,
Q represents a bond,
* is the linkage site to the residue represented by A, and
l represents 0
and the salts thereof, the solvates thereof and the solvates of the salts thereof, in combination with at least one β-lactamase inhibitor (BLI) selected from the group comprising clavulanic acid, tazobactam, sulbactam and other BLIs belonging to the groups of lactam inhibitors, diazabicyclooctane inhibitors, transition state analog inhibitors and/or metallo-β-lactamase inhibitors
and
at least one additional β-lactamase selected from the groups of class C AmpC β-lactamases and/or class A, class B, class C and/or class D carbapenemases.

2. A composition as defined in claim 1, wherein the compound of formula I is selected from the group comprising compounds of formulae (I-a), (I-b), (I-c), (I-d), (I-e), (I-f), and (I-g),

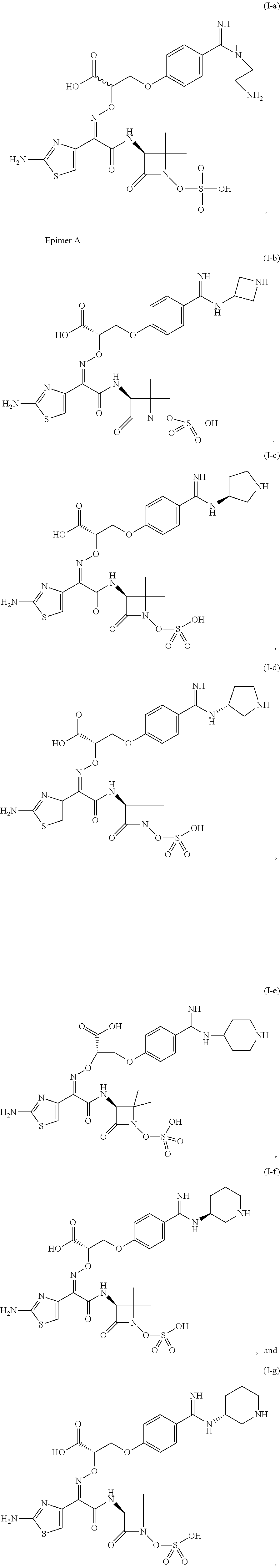

3. A composition as defined in claim 1 wherein the BLI is selected from the group consisting of clavulanic acid, tazobactam, and sulbactam.

4. A composition as defined in claim 1, wherein the BLI is selected from the group consisting of clavulanic acid, tazobactam, and sulbactam, and wherein the dose of clavulanic acid is 0.1-0.6 g, wherein the dose of tazobactam is 0.1-10 g, and wherein the dose of sulbactam is 0.25 g to 4.0 g.

5. A composition as defined in claim 1, wherein the BLI is selected from the group consisting of clavulanic acid, tazobactam, and sulbactam, and wherein the dose of clavulanic acid is 0.25-0.6 g, wherein the dose of tazobactam is 0.25-2.0 g, and wherein the dose of sulbactam is 0.25 to 2.0 g.

6. A composition as defined in claim 1, wherein the BLI is selected from the group consisting of clavulanic acid, tazobactam, and sulbactam, and wherein the dose of the compound of formula (I) and the BLI are administered at a ratio selected from the following group of ratios: 100:1 to 1:100, 10:1 to 1:10, and 5:1 to 1:5.

7. A composition as defined in claim 2, wherein the compound is a compound of formula (I-g).

8. A method for the treatment or prophylaxis of a subject having an infection caused by Gram-negative bacteria that produce at least one or more class A and/or class D extended-spectrum β-lactamase (ESBL) which comprises administering to the subject a composition of claim 1.

9. The method of claim 8, wherein the Gram-negative bacteria are selected from the genus of Enterobacteriaceae and non-fermenting strains.

10. A composition as defined in claim 2, wherein the BLI is selected from the group consisting of clavulanic acid, tazobactam, and sulbactam.

11. A composition as defined in claim 2, wherein the BLI is selected from the group consisting of clavulanic acid, tazobactam, and sulbactam, and wherein the dose of clavulanic acid is 0.1-0.6 g, wherein the dose of tazobactam is 0.1-10 g, and wherein the dose of sulbactam is 0.25 g to 4.0 g.

12. A composition as defined in claim 2, wherein the BLI is selected from the group consisting of clavulanic acid, tazobactam, and sulbactam, and wherein the dose of clavulanic acid is 0.25-0.6 g, wherein the dose of tazobactam is 0.25-2.0 g, and wherein the dose of sulbactam is 0.25 to 2.0 g.

13. A composition as defined in claim 2, wherein the BLI is selected from the group consisting of clavulanic acid, tazobactam, and sulbactam, and wherein the dose of the compound of formulae (I-a) to (I-g) and the BLI are administered at a ratio selected from the following group of ratios: 100:1 to 1:100, 10:1 to 1:10, and 5:1 to 1:5.

14. A method for the treatment or prophylaxis of a subject having an infection caused by Gram-negative bacteria that produce at least one or more class A and/or class D extended-spectrum β-lactamase (ESBL) which comprises administering to the subject a composition of claim 2.

15. The method of claim 14, wherein the Gram-negative bacteria are selected from the genus of Enterobacteriaceae and non-fermenting strains.
